(19) United States
(12) Patent Application Publication
(10) Pub. No.: US 2015/0336882 A1
(43) Pub. Date: Nov. 26, 2015

(54) PHOTOCHEMICAL PROCESS FOR THE PREPARATION OF 3-((3,3,3-TRIFLUOROPROPYL)THIO)PROPIONIC ACID AND ESTERS THEREOF

(71) Applicant: Dow AgroSciences LLC, Indianapolis, IN (US)

(72) Inventors: Kaitlyn GRAY, Freeland, MI (US); Yu ZHANG, Carmel, IN (US); Beth LORSBACH, Indianapolis, IN (US)

(21) Appl. No.: 14/517,340

(22) Filed: Oct. 17, 2014

Related U.S. Application Data
(60) Provisional application No. 62/001,921, filed on May 22, 2014.

(51) Int. Cl. C07C 323/52

(52) U.S. Cl. CPC C07C 323/52

(57) ABSTRACT

3-((3,3,3-Trifluoropropyl)thio)propionic acid and esters thereof are prepared by a photochemical process involving the free radical coupling of 3-mercaptopropionic acid and esters thereof with 3,3,3-trifluoropropene. The ratio of linear to branched isomer is enhanced.

### CROSS REFERENCE TO RELATED APPLICATIONS

This application claims the benefit of U.S. Provisional Patent Application Ser. No. 62/001,921, filed May 22, 2014, the entire disclosure of which is hereby expressly incorporated by reference into this Application.

### TECHNICAL FIELD

The present invention concerns a process for the preparation of 3-((3,3,3-trifluoro-propyl)thio)propionic acid and esters thereof. More particularly, the present invention concerns a photochemical process for the preparation of 3-((3,3,3-trifluoropropyl)thio)-propionic acid and esters thereof by the free radical coupling of 3-mercaptopropionic acid and esters thereof with 3,3,3-trifluoropropene.

### BACKGROUND

3-((3,3,3-Trifluoropropyl)thio)propionic acid and methyl 3-((3,3,3-trifluoropropyl)-thio)propionate are useful to produce pesticidal thioether and pesticidal sulfoxides such as N-(3-chloro-1-(pyridin-3-yl)-1H-pyrazol-4-yl)-N-ethyl-3-((3,3,3-trifluoropropyl)thio)- propanamide

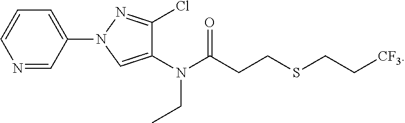 [SMILES: CCN(C(=O)CCSCCC(F)(F)F)c1cn(-c2cccnc2)nc1Cl]

Methyl 3-((3,3,3-trifluoropropyl)thio)propionate has typically been produced by the free radical addition of methyl 3-mercaptopropionate to 3,3,3-trifluoropropene in the presence of a free radical initiator such as a-azobisisobutyronitrile (AIBN). The ratio of the desired linear isomer to the undesired branched isomer is approximately 10:1.

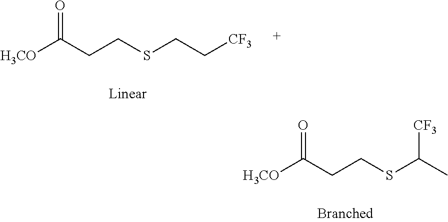

It would be desirable to more selectively make methyl 3-((3,3,3-trifluoropropyl)thio)-propionate or 3-((3,3,3-trifluoropropyl)thio)propionic acid with less of the branched isomer.

### SUMMARY

The present invention concerns a process for the preparation of 3-((3,3,3-trifluoro-propyl)thio)propionic acid or esters thereof by the photochemical coupling of 3-mercapto-propionic acid or esters thereof with 3,3,3-trifluoropropene. More particularly, the present invention concerns a process for the preparation of 3-((3,3,3-trifluoropropyl)thio)propionic acid or esters thereof (Formula I)

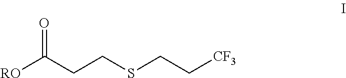

wherein R represents H or C₁-C₄ alkyl

which comprises photochemically coupling 3-mercaptopropionic acid or esters thereof (Formula II)

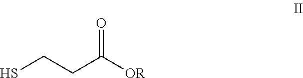

wherein R is as previously defined

with 3,3,3-trifluoropropene (Formula III)

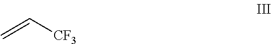

in the presence of 2,2-dimethoxy-2-phenylacetophenone initiator and long wavelength UV light in an inert organic solvent.

### DETAILED DESCRIPTION OF DISCLOSURE

As used herein, the term “alkyl” denotes branched or unbranched hydrocarbon chains. The photochemical coupling of the present invention more selectively prepares 3-((3,3,3-trifluoropropyl)thio)propionic acid or esters thereof. The ratio of linear to branched isomer is enhanced from about 10:1 to about 20:1 or greater.

While stoichiometric amounts of 3-mercaptopropionic acid or esters thereof and 3,3,3-trifluoropropene are required, because of its low boiling point, excess 3,3,3-trifluoropropene is usually employed to compensate for routine losses.

From about 1 to about 10 mole percent initiator, 2,2-dimethoxy-2-phenyl-acetophenone, is typically used, with about 5 mole percent being preferred.

Long wavelength UV light is sometimes called “black light” and ranges from about 400 to about 365 nanometers.

The photochemical coupling is conducted in an inert organic solvent. Typical inert organic solvents must remain liquid to −70° C., must remain relatively inert to the free radical conditions and must dissolve the reactants at reaction temperatures. Preferred inert organic solvents are aromatic and aliphatic hydrocarbons like toluene.

The temperature at which the reaction is conducted is not critical but usually is from about −70° C. to about 35° C. Initially, it is important to keep the temperature below the boiling point of 3,3,3-trifluoropropene, i.e., about −18 to −16° C.

In a typical reaction, the inert organic solvent is cooled to less than about −50° C. and the 3,3,3-trifluoropropene is bubbled into the solvent. The 3-mercaptopropionic acid or esters thereof and 2,2-dimethoxy-2-phenylacetophenone are added and a long wave function (366 nm) UVP lamp (4 watt) is turned on. After sufficient conversion of 3-mercapto-propionic acid or esters thereof, the light is turned off and the solvent removed.

The following examples are presented to illustrate the invention.

### EXAMPLES

Weight percent purities were determined using a GC internal standard assay with octanophenone as the internal standard. Linear/branched ratios are based on GC area percent of the respective linear and branched products. GC Method Details: Agilent DB-5MS (122-5532) column 30 m×0.25 mm×0.25 um; heater: 250° C.; control mode, flow: 2 mL/min; oven program: 50° C. for 2 min then 20° C./min to 280° C. for 8 min.

### 1. Photochemical Synthesis of Methyl 3-((3,3,3-Trifluoropropyl)Thio)Propionate

A 500 mL three-neck round bottomed flask was charged with toluene (200 mL) and cooled to <−50 ° C. with a dry ice/acetone bath. 3,3,3-Trifluoropropene (21.8 g, 227 mmol) was condensed into the reaction by bubbling the gas through the cooled solvent and the ice bath was removed. Methyl 3-mercaptopropionate (26.8 g, 223 mmol) and 2,2-dimethoxy-2-phenylacetophenone (2.72 g, 10.61 mmol) were added and a UVP lamp (4 watt) that was placed within 2 centimeters of the glass wall was turned on to the long wave function (366 nanometers). The reaction reached 35° C. due to heat from the lamp. After 4 hours, all of the trifluoropropene was either consumed or boiled out of the reaction. The light was turned off and the reaction stirred at room temperature overnight. After 22 hours, more trifluoropropene (3.1 g) was bubbled through the mixture at room temperature and the light was turned on for an additional 2 hours. The reaction had converted 93% so no more trifluoropropene was added. The light was turned off and the mixture concentrated on the rotovap (40° C., 20 torr) giving a yellow liquid (45.7 g, 21.3:1 linear : branched isomer, 75 wt % pure linear isomer determined by a GC internal standard assay, 34.3 g active, 71% in pot yield): ¹H NMR (400 MHz, CDCl₃) δ 3.71 (s, 3H), 2.82, (td, J=7.3, 0.7 Hz, 2H), 2.75-2.68 (m, 2H), 2.63 (td, J=7.2, 0.6 Hz, 2H), 2.47-2.31 (m, 2H); ¹³C NMR (101 MHz, CDCl₃) δ 172.04 , 125.93 (q, J=277.2 Hz), 51.86, 34.68 (q, J=28.6 Hz), 34.39, 27.06, 24.11 (q, J=3.3 Hz); ¹⁹F NMR (376 MHz, CDCl₃) δ-66.53.

### 2. Free Radical Initiator Synthesis of Methyl 3-((3,3,3-Trifluoropropyl)Thio)Propionate

A 2 L autoclave reactor was charged with toluene (716.45 g), methyl 3-mercapto-propionate (187.78 g, 1562.6 mmol), and a-azobisisobutyronitrile (12.890 g, 78.50 mmol). The reactor was sealed and pressurized with nitrogen (˜100 psig) three times to purge the system of air. 3,3,3-Trifluoropropene (153.20 g, 1595.0 mmol) was added via transfer cylinder at 12° C. (cold water bath). The reaction was heated to 80 ° C. and stirred for 21 hours. The reaction was allowed to cool to room temperature and vacuum transferred out of the reactor. The crude solution was concentrated by rotary evaporation (bath: 40° C., 12 mm Hg) to providing a clear yellow liquid (371.95 g, 9.8:1 linear : branched isomer, 69 wt % pure linear isomer determined by a GC internal standard assay, 257.39 g active, 76% in pot yield).

### 3. Photochemical Synthesis of 3-((3,3,3-Trifluoropropyl)Thio)Propanoic Acid

A 250 mL three-neck round bottomed flask was charged with toluene (81 mL) and cooled to <−50 ° C. with a dry ice/acetone bath. 3,3,3-Trifluoropropene (10.28 g, 107.0 mmol) was bubbled into the solvent and the ice bath was removed. 3-Mercaptopropionic acid (9.200 g, 86.70 mmol) and 2,2-dimethoxy-2-phenylacetophenone (1.070 g, 4.170 mmol) was added and the long wave light (366 nm, 4 watt UVP lamp) was turned on (Starting temperature: −24° C.). The reaction reached a high temperature of 27.5° C. due to heat from the lamp. The reaction was stirred with the black light on for 4 hours. After 4 hours the black light was turned off and the reaction concentrated by rotary evaporation (41° C., 6 mm Hg) giving a pale yellow oil (18.09 g, 51:1 linear:branched isomer, 90 wt % linear isomer by GC internal standard assay, 16.26 g active, 93%). The crude material was dissolved in 10 wt % sodium hydroxide w/w (37.35 g) and was washed with toluene (30 mL) to remove non-polar impurities. The aqueous layer was acidified to pH ˜2-3 with hydrochloric acid (2 N, 47.81 g) and was extracted with toluene (50 mL). The organic layer was washed with water (40 mL) and dried over magnesium sulfate, filtered, and concentrated by rotary evaporation giving a pale yellow oil (14.15 g, 34:1 linear:branched isomer, 94 wt % linear isomer by GC internal standard assay, 13.26 g active, 76%): ¹H NMR (400 MHz, CDCl₃) δ 2.83 (td, J=7.1, 0.9 Hz, 2H), 2.76-2.64 (m, 4H), 2.47-2.30 (m, 2H); ¹³C NMR (101 MHz, CDCl₃) δ 177.68, 125.91 (q, J=277.1 Hz), 34.58 (q, J=28.8 Hz), 34.39, 26.63, 24.09 (q, J=3.3 Hz); ¹⁹F NMR (376 MHz, CDCl₃) δ-66.49.

4. Free radical initiator synthesis of 3-((3,3,3-trifluoropropyl)thio)propanoic acid:

A 100 mL stainless steel Parr reactor was charged with azobisisobutyronitrile (AIBN, 0.231 g, 1.41 mmol), toluene (45 mL), 3-mercaptopropionic acid (3.40 g, 32.0 mmol), and octanophenone (526.2 mg) as an internal standard and was purged and pressure checked with nitrogen. The reactor was cooled with dry ice and the 3,3,3-trifluoropropene (3.10 g, 32.3 mmol) was condensed into the reactor. The ice bath was removed and the reactor heated to 60° C. and stirred for 27 hours. The internal yield of the reaction was determined to be 80% by use of the octanophenone internal standard (12.2:1 linear:branched isomer). The pressure was released and the crude mixture removed from the reactor. The mixture was concentrated by rotary evaporation and sodium hydroxide (10%, 50 mL) was added. The solution was washed with methyl tent-butyl ether (50 mL) then acidified to pH ˜1 with hydrochloric acid (6 N). The product was extracted with 100 mL methyl tert-butyl ether, dried over magnesium sulfate, filtered, and concentrated to give the crude titled compound as an oil (5.34 g, 11.9:1 linear:branched isomer, 88 area% pure linear isomer by GC).

1. A process for the preparation of 3-((3,3,3-trifluoropropyl)thio)propionic acid or esters thereof (Formula I) 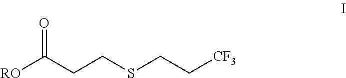
wherein R represents H or C₁-C₄ alkyl
which comprises photochemically coupling 3-mercaptopropionic acid or esters thereof (Formula II) 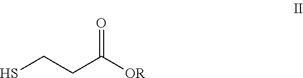
wherein R is as previously defined
with 3,3,3-trifluoropropene (Formula III) 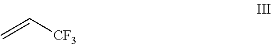
in the presence of 2,2-dimethoxy-2-phenylacetophenone initiator and long wavelength UV light in an inert organic solvent.

2. The process of claim 1, wherein the step of photochemically coupling is initiated at a temperature below −16 ° C.

3. The process of claim 1, wherein the ratio of Formula I to an undesired branched product is >20:1.
